(19) United States
(12) Patent Application Publication
(10) Pub. No.: US 2012/0271129 A1
(43) Pub. Date: Oct. 25, 2012

(54) MULTI-FREQUENCY RF MODULATED NEAR INFRARED SPECTROSCOPY FOR HEMOGLOBIN MEASUREMENT IN BLOOD AND LIVING TISSUES

(76) Inventor: Ming Wang, San Jose, CA (US)

(21) Appl. No.: 13/452,847

(22) Filed: Apr. 21, 2012

Related U.S. Application Data
(60) Provisional application No. 61/478,443, filed on Apr. 22, 2011.

(51) Int. Cl. A61B 5/1455

(52) U.S. Cl. 600/323

(57) ABSTRACT

The present application discloses a tissue oximeter system which includes radio frequency (RF) wave sources configured to produce RF waves at different RF frequencies, near infrared (NIR) light sources each configured to emit NIR lights each modulated by one or more of the RF waves generated by the RF wave sources, an optical probe that directs the NIR lights modulated at different RF frequencies to a living tissue, and wherein the optical probe includes a plurality of light-emitting points that each can couple one of the NIR lights into the living tissue, one or more optical detectors that each can receive scattered lights from the living tissue and to convert the scattered lights into electronic signals, and a control and data acquisition unit that can calculate absolute level of [HbO], [Hb], or [SO2] based on the electronic signals.

### PRIORITY CLAIM AND RELATED PATENT APPLICATION

This patent document claims priority to U.S. Provisional Application No. 61/478,443 entitled “Multiple RF modulation (MRFM) scheme and MRFM-based NIR spectroscopy featuring self calibration for accurate and valid hemoglobin measurement of blood and living tissues” and filed Apr. 22, 2011 by the same inventor, the disclosures of which is incorporated by reference as part of the disclosure of this document.

### BACKGROUND OF THE INVENTION

The present application relates to near infrared (NIR) spectroscopy based hemoglobin meters and tissue oximeters.

NIR spectroscopy, also referred as spectrophotometry, has for decades been applied to real time non-invasive measurements of biological properties of arterial blood and living tissues, and to assessing and diagnosing physiological condition of a patient. One important such application is tissue oximeter that detects concentrations of individual chromophores using NIR lights at multiple wavelengths. The chromophores include as oxygenated hemoglobin (i.e. oxyhemoglobin) and deoxygenated hemoglobin (i.e. deoxyhemoglobin) in blood and living tissues.

NIR spectroscopy based tissue oximeter delivers NIR lights into a living tissue or blood, and detects NIR lights coming out of the living tissue to generate biological information about the living tissue. The biological information includes oxyhemoglobin concentration [HbO], deoxyhemoglobin concentration [Hb], and oxygen saturation [SO2]. [SO2] is the ratio of [HbO] to total hemoglobin [HbT], that is, the sum of [HbO] and [Hb].

Tissue oximeters can measure relative or absolute oxygen saturations. Relative oxygen saturation presents a trend of [SO2] change over time based on relative concentrations of [HbO] and [Hb]. Absolute oxygen saturation captures the true value of [SO2] based on the absolute [HbO] and [Hb] values. The measurement of relative or absolute oxygen saturation can be determined by controlling NIR light sources in tissue oximeters.

Generally, a tissue oximeter having a continuous wave light source can generate relative oxygen saturation, whereas a tissue oximeter a radio frequency (RF) modulated light source can generate absolute oxygen saturation.

Accurate readings of [HbO], [Hb], and [SO2] are crucial parameters for surgeons, physicians, and healthcare givers to provide diagnosis and intensive care to patients. Although tissue oximeters having RF modulated NIR light sources can produce absolute measurements for [HbO], [Hb] and [SO2], there are still issues related to the accuracies and precisions of the measurements generated by such tissue oximeters. One significant measurement inaccuracy arises from inhomogeneity in living tissues. The measured [HbO] or [Hb] values often vary depending the structure and density of the living tissues in the portion of the patient's body that is being measured.

There is therefore a need for a tissue oximeter that can conduct more accurate and precise measurements of oxyhemoglobin concentration, deoxyhemoglobin concentration, and oxygen saturation in blood and living tissues.

### SUMMARY OF THE INVENTION

The present application discloses methods, apparatus, systems, algorithm, and related computer programs for real time and non-invasive detecting, measuring and monitoring of hemoglobin concentration, and oxygen saturation of hemoglobin in blood, and living tissues which includes the brain. The disclosed methods and systems can significantly improve the accuracy and quality of the clinic and pathological assessments and diagnosis of the physiological conditions of patients by doctors, healthcare givers, and the patients themselves.

Specifically, the present application discloses a tissue oximeter based on RF modulated NIR spectroscopy. The light source in such a tissue oximeter is modulated by electronic waveforms at different frequencies. Examples of such electronic waveforms include sinusoidal and square waves. NIR spectroscopy with multiple RF modulation disclosed in the present application can use fewer optical paths, and can calibrate manufacturing-related variability and errors in mechanical, electronic and optical parameters in a single optical path, which result in higher accuracy and smaller size of optical probes compared to conventional systems. Furthermore, smaller probes allow more applications to clinics, assessments and healthcares, too.

In one general aspect, the present invention relates to a tissue oximeter system that includes N number of radio frequency (RF) wave sources that can produce RF waves at different RF frequencies, wherein N is an integer bigger than 1; M number of near infrared (NIR) light sources that each can emit NIR lights each modulated by one or more of the RF waves generated by the N number of RF wave sources, wherein M is an integer bigger than 1; an optical probe that can direct the NIR lights modulated at different RF frequencies to a living tissue, and wherein the optical probe comprises a plurality of light-emitting points that each can couple one of the NIR lights into the living tissue; one or more optical detectors that each can receive scattered lights from the living tissue and to convert the scattered lights into electronic signals; and a control and data acquisition unit that can calculate absolute level of [HbO], [Hb], or [SO2] based on the electronic signals.

Implementations of the system may include one or more of the following. The tissue oximeter system can further include an optical fiber bundle comprising a plurality of optical fibers that each can deliver one of the NIR lights to the plurality of light-collecting points on the optical probe. The tissue oximeter system can further include an optical multiplexer that can direct the NIR lights generated by the M number of NIR light sources to the optical probe; and one or more optical fibers that each can deliver one of the NIR lights from the optical multiplexer to the optical probe. The optical multiplexer can direct different

NIR lights through one of the optical fibers at different times. The optical probe can collect scattered lights from the living tissue at multiple locations on the living tissue, wherein the scattered lights have travelled through different optical paths in the living tissue. The optical probe can include a plurality of light-collecting points that can collect the scattered lights at the multiple locations on the living tissue. There can be a different number of the optical detectors from the M number of NIR light sources. There can be M number of the optical detectors.

In one general aspect, the present invention relates to a tissue oximeter system that includes a plurality of radio frequency (RF) wave sources that can genarate RF waves at different RF frequencies; one or more near infrared (NIR) light sources that each can emit NIR lights each modulated by one or more the RF waves at different RF frequencies; an optical probe that can direct the NIR lights modulated at different RF frequencies to a living tissue; one or more optical detectors that can receive scattered lights from the living tissue and to convert the scattered lights into electronic signals; and a control and data acquisition unit that can calculate absolute level of [HbO], [Hb], or [SO2] based on the electronic signals.

Implementations of the system may include one or more of the following. The scattered lights may have travelled in different optical paths in the living tissue. The optical probe can include a plurality of light-emitting points that each can couple one of the NIR lights into the living tissue. The optical probe can include a plurality of light-collecting points that can collect the scattered lights at the multiple locations on the living tissue. The scattered lights may have travelled in single optical path in the living tissue. The optical probe can include a single light-emitting point that can couple each of the NIR lights into the living tissue and a single light-collecting point that can collect the scattered lights after the NIR lights travels through the single optical path.

In one general aspect, the present invention relates to a method for calibrating a tissue oximeter system. The method includes modulating a first near infrared (NIR) light at a first radio frequency at a first light source; introducing the first NIR light in a single optical path in a living tissue; measuring intensity and phase of the first NIR light after first NIR light travels through the single optical path in the living tissue; modulating a second NIR light at a second radio frequency at a second light source; introducing the second NIR light in the same single optical path in the living tissue; measuring intensity and phase of the second NIR light after second NIR light travels through the single optical path in the living tissue; and calculating absorption coefficient and scattering coefficient of the living tissue using the intensities and the phases of the first second NIR light and the second NIR light.

Implementations of the system may include one or more of the following. The method can further include calculating total hemoglobin and oxygenation of the living using the absorption and scattering coefficients. The absorption coefficient and the scattering coefficient tissue can be associated with the optical path in the living tissue. The step of calculating can include calculating a ratio of the light intensities of the first NIR light and the second NIR light measured respectively after the first NIR light and the second NIR light have travelled through the single optical path in the living tissue. The step of calculating can include calculating a phase difference between the light intensities of the first NIR light and the second NIR light measured respectively after the first NIR light and the second NIR light have travelled through the single optical path in the living tissue. The single optical path is defined by a single light entry point and a single light exit point on the living tissue.

These and other aspects, their implementations and other features are described in detail in the drawings, the description and the claims.

### BRIEF DESCRIPTION OF THE DRAWINGS

FIG. 1 is a block diagram for a tissue oximeter system in accordance with the present invention.

FIG. 2 is a schematic diagram illustrating the details of the multi-frequency RF modulation unit in the tissue oximeter system shown in FIG. 1.

FIG. 3A shows an example of multiple optical paths for the NIR lights into living tissue in accordance with the present invention.

FIG. 3B shows an exemplified configuration of optical probe capable of emitting RF modulated NIR lights at different RF frequencies at different positions and collecting scattered NIR lights at multiple locations in accordance with the present invention.

FIG. 4 illustrates an exemplified flow chart for the operation of a multi-frequency RF modulated tissue oximeter in accordance with the present invention.

FIG. 5 shows an example of single optical path for the NIR lights into living tissue in accordance with the present invention.

FIG. 6 illustrates an exemplified flow chart for self-calibration of the tissue oximeter system in accordance with the present invention.

### DETAILED DESCRIPTION OF THE INVENTION

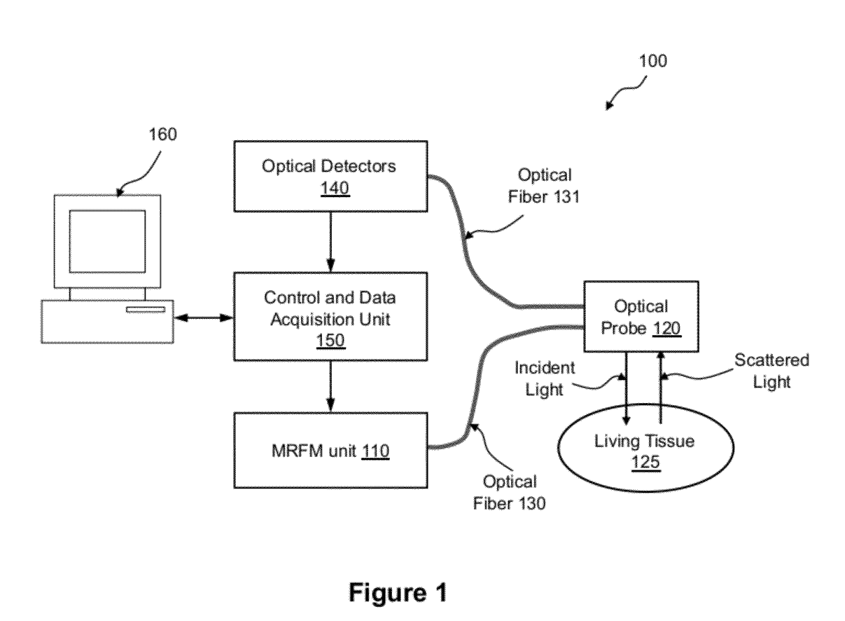

Referring to FIG. 1, a tissue oximeter system 100 includes a multi-frequency RF modulation (MRFM) unit 110, an optical probe 120, one or more optical detectors 140, and a control and data acquisition unit 150. The optical probe 120 is connected with the MRFM unit 110 and the optical detector 140 by optical fibers 130, 131. The MRFM unit 110, as described in more detail below, can produce RF modulated NIR light at multiple radio frequencies. The NIR light is transmitted in the optical fiber 130 to the optical probe 120, which in turn delivers the NIR light (i.e. incident light) into an living tissue 125 or blood (not shown) of a patient. The living tissue can include brain tissues in a patient's head. The optical probe 120 also collects NIR light scattered by the living tissue or blood of the patient, and transmit the collected NIR light to the optical detector 140 via the optical fiber 131. The collected optical signals reflect optical properties of the hemoglobin in the living tissue 125 and carries biological information about the hemoglobin. The biological information includes oxyhemoglobin concentration [HbO], deoxyhemoglobin concentration [Hb], and oxygen saturation [SO2]. The optical detector 140 converts the collected NIR light or optical signals into electronic signals, which are in turn analyzed by the control and data acquisition unit 150. The control and data acquisition unit 150 can demodulate the electronic signals from the modulating RF frequencies, and extract [HbO] and [Hb] in the living tissue 125 from the electronic signals. The control and data acquisition unit 150 can obtain absolute concentration levels of oxyhemoglobin and deoxyhemoglobin.

The tissue oximeter system 100 can also include a computer 160 in communication with the control and data acquisition unit 150. The computer 160 can be standalone or embedded as a part of another unit or module such as the control and data acquisition unit 150. The computer 160 can compute [SO2] based on the measured [HbO] and [Hb] values. The tissue oximeter system 100 can obtain absolute oxygen saturation [SO2] in the living tissue 125 or blood of the patient.

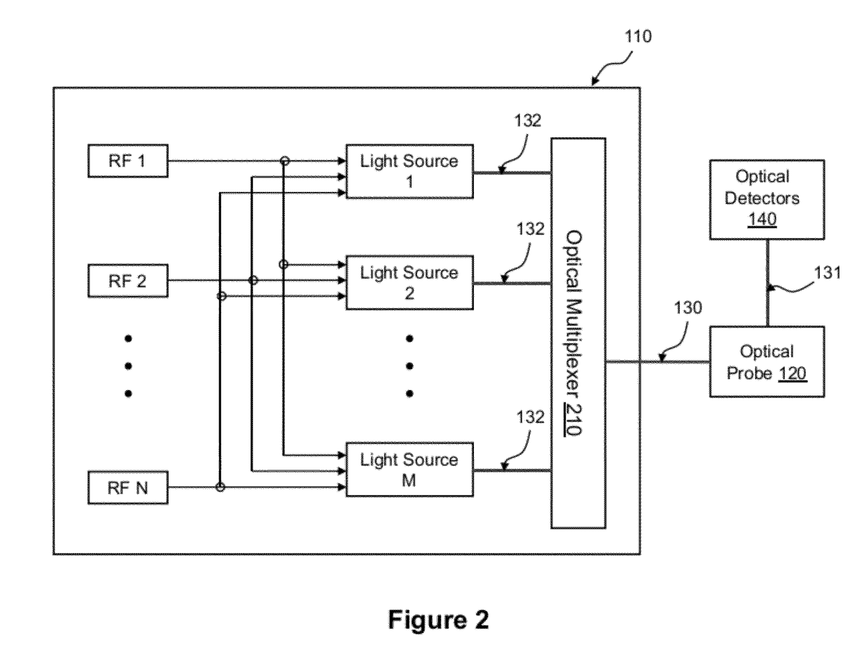

Referring to FIG. 2, the MRFM unit 110 includes a plurality of RF wave sources (i.e. RF oscillators) RF 1, RF 2 . . . RF N, wherein Nis an integer bigger than 1. The RF wave sources RF 1, RF 2 . . . RF N can modulate one (M=1) or multiple NIR light sources 1, 2 . . . M (M>2) to produce NIR lights modulated by RF signals at different radio frequencies f₁, f₂, . . . F_{M}.

Each of the M NIR light sources 1-M is modulated by all of the N RF waves. Each of the M RF modulated NIR lights is coupled via a separate optical fiber from the corresponding NIR light source to an optical multiplexer 210. The optical multiplexer 210 can direct each of the M RF modulated NIR lights to the optical probe 120 (and thus the living tissue 125) via the optical fiber 130. There can be one or multiple optical fibers 130 in a fiber bundle connecting between the optical multiplexer 210 and the optical probe 120. The different RF modulated NIR lights are transmitted at different time periods (i.e. time division) through one of the optical fibers 130.

To obtain the total hemoglobin and absolute value of oxygen saturation of hemoglobin of living tissue of human being and animals, both absorption and scattering coefficients are needed for deoxygenated and oxygenated hemoglobin. Continuous-wave (CW) NIRS based tissue oximeters calculate oxygen saturation from the measured absorption coefficient only, under the hypothetical assumption that the scattering coefficients of the tissue to NIR lights are the same and constant. This assumption is not true in living tissues and as a result CW NIRS based tissue oximeters often produces inaccurate and incorrect results. The RF scheme technology can help to solve the problem by collecting both absorption and scattering coefficients of hemoglobin to the NIR lights. The RF modulation technology extracts both absorption and scattering coefficients from the measured data of intensity attenuation (d.c. and a.c. components) as well as the RF phase shift happened when photons migrate through the tissue.

The phases of the N RF waves shift with the optical path which the intensity modulated light traveled through the tissue under measurement. The RF phase shifts and the demodulation of modulated light are used to calculate the optical property of tissue under measurement for the biological parameters such as total hemoglobin and the tissue oxygenation.

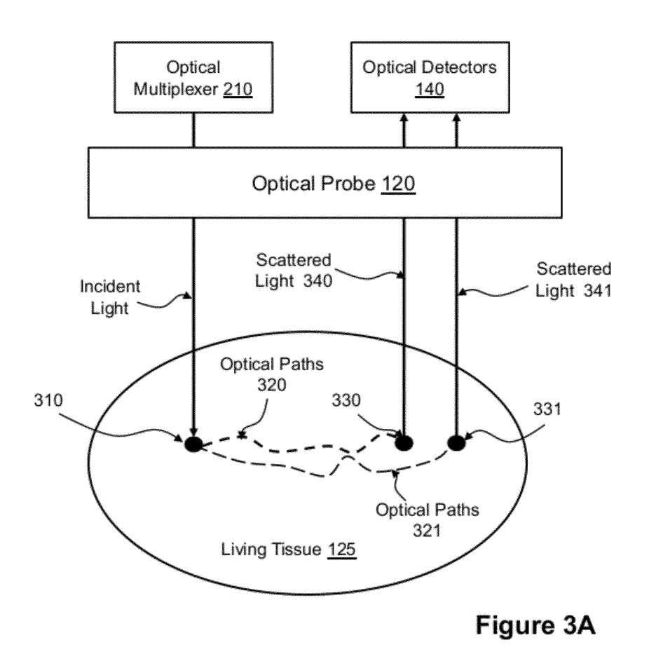

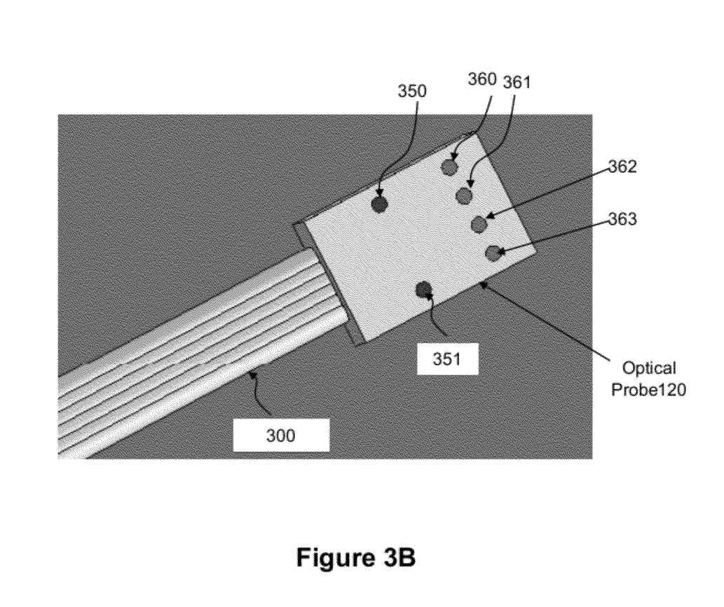

The tissue oximeter system can obtain absolute value of oxyhemoglobin and deoxyhemoglobin based on measurement of the RF frequency dependent phase shift and demodulation of intensity modulated NIR lights that have passed through the blood and/or living tissues. To quantitatively measure optical absorption of oxygenated and deoxygenated hemoglobin to the NIR light, the optical path which the NIR light traveled thought the tissue under measurement must be determined correctly. Basically, optical property of tissue includes both absorption and scattering to the NIR lights traveling though the tissue. Because of the scattering effect of tissue on the lights, the optical path is not simply the geometrical separation between the light source and the optical detector, as shown in FIGS. 3A and 3B. The optical path changes with different tissue under measurement. Such path changes affect precision and accuracy of the acquired optical properties and consequently affect the accuracy and precision of extracted bio-information about hemoglobin of tissue under measurement. Oximeters which measure absorption only cannot quantitatively extract correct information about hemoglobin and oxygenation.

RF modulation can help to measure the optical paths precisely and therefore obtain correct information about hemoglobin and oxygenantion. In an NIRS with light sources modulated by a RF wave, the RF wave will have a phase shift when the NIR light traveled through the tissue under measurement. This phase shift relates to the real optical path. It has been proven and accepted to tissue with high scattering that the quantitative hemoglobin and oxygenation can be extracted from the acquired phase shift information and demodulation, where the demodulation is defined as ratio of detected AC component to detected DC component divided by ratio of emitted AC component to emitted DC component from laser source.

The presently disclosed tissue oximeter system 100 is compatible with a single or multiple optical paths in living tissues. In some embodiments, the presently disclosed tissue oximeter system 100 uses multiple optical paths in the NIRS and the tissue oximeters to reduce effects of uncertainty of light source intensity, detector sensitivity and light coupling efficiency, etc., on data accuracy which leads to the accuracy and precision of the total hemoglobin and oxygen saturation. Multiple optical paths are particularly useful for detecting and extracting bio-information over a large area of tissues such as big brain area, kidney, lung, liver, etc., and for constructing 2D (2-dimensional) and 3D image of tumor (such as breast cancer) or stroke, etc.

Referring to FIG. 3A, the optical probe 120 guides NIR light each modulated by an RF wave at a frequency to living tissue 125 at the light entry point 310. The NIR light is scattered and travels in the living tissue 125 in different optical paths 320, 321. The scattered lights 340, 341 are then respectively collected at different light exit points 330, 331. The scattered lights 340, 341 are then transmitted by the optical probe 120 and captured by different optical detectors 140. Similarly, multiple optical paths can be formed between multiple entry points and a single light exit point, or between multiple entry points and multiple light exit points.

FIG. 3B shows an exemplified configuration of optical probe 120 capable of emitting RF modulated NIR lights at different RF frequencies at different positions and collecting scattered NIR lights at multiple locations. The optical probe 120 is connected to an optical fiber bundle 200, which includes the optical fibers 130, 131 (FIG. 2). The optical probe 120 receives NIR lights modulated at different radio frequencies from the optical fibers 130, and emits the NIR lights at light-emitting points 350, 351 that are in contact with the living tissue 125 at the light entry points 310 (FIG. 3A). The light-emitting points 350, 351 can be tips of the optical fibers 130 for guiding the NIR lights. Each light-emitting point 350 or 351 emits two or more NIR light each at a different RF frequency. The optical probe 120 also includes light-collecting points 360-363 which are in contact with the living tissue 125 at the light exit points 330-331, etc. The scattered lights emitted at different light emitting points 350, 351 and collected at different light-collecting points 360-363 have travelled in the living tissue 125 through different optical paths. For example, in the specific example shown in FIG. 3B, there are 2 light emitting points and 4 light collection points so there are 2×4=8 optical paths.

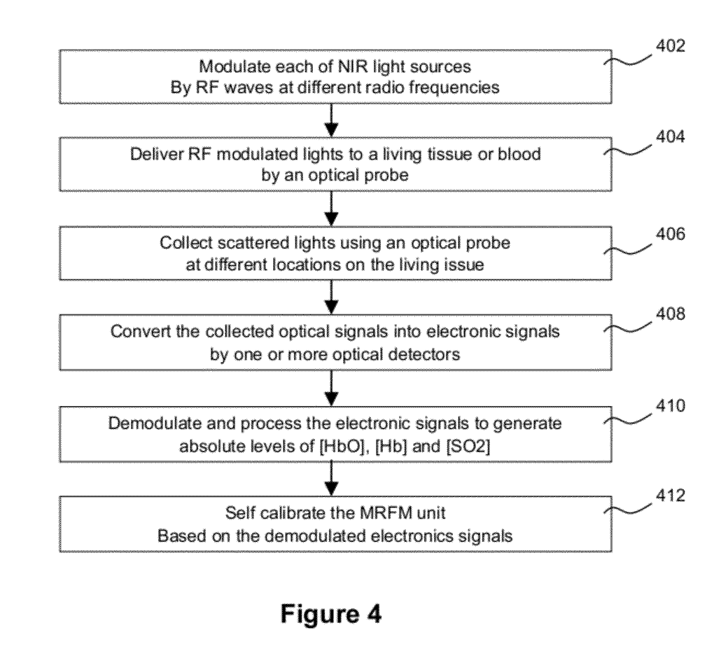

In some embodiments, the tissue oximeter system 100 is capable of self-calibration to generate accurate, precise and valid biological properties of blood and living tissue under measurement. Referring to FIG. 4, the self-calibration method can include one or more of the following steps: First, each of one or more light sources is modulated by multiple RF waves at different radio frequencies (step 402). The RF modulated NIR light is delivered to a living tissue or blood of a patient (step 404). The NIR lights that have passed through the living tissue or blood are collected at different locations of the living tissue (step 406). The scattered NIR lights collected at different locations have travelled through different optical paths (as shown in FIGS. 3A and 3B). The collected optical signals are converted to electronics signals by one or more optical detectors (step 408). The electronic signals are demodulated and processed to generate absolute levels of [HbO], [Hb] and [SO2] (step 410). The demodulation of the electronic signals carrying the multiple RF waves at different frequencies allow the absolute values of [HbO], [Hb] and [SO2] in the living tissue or the blood of the patient, which are more precise and accurate than some conventional systems.

In according to the present invention, the MRFM can be self calibrated using the demodulated electronic signals (step 412). Many factor can affect the accuracies of detected signals using RF modulated NIR light sources: for example, variation of intensity and phase of light sources, differences in coupling efficiencies between light source and tissue and between tissue and optical detector, differences in of sensitivities of optical detectors, manufacturing errors in the distances between light source and optical detectors, the optical inhomogeneity of tissue to the NIR lights, etc. Multiple optical paths can differentiate the optical properties of hemoglobin in blood and/or in living tissues. Inhomogeneities in the living tissue 125 as well as errors and/or deviations in the optical paths caused during manufacturing process may lead to measurement accuracy and validity that can severely impact clinic practices.

Self-Calibration

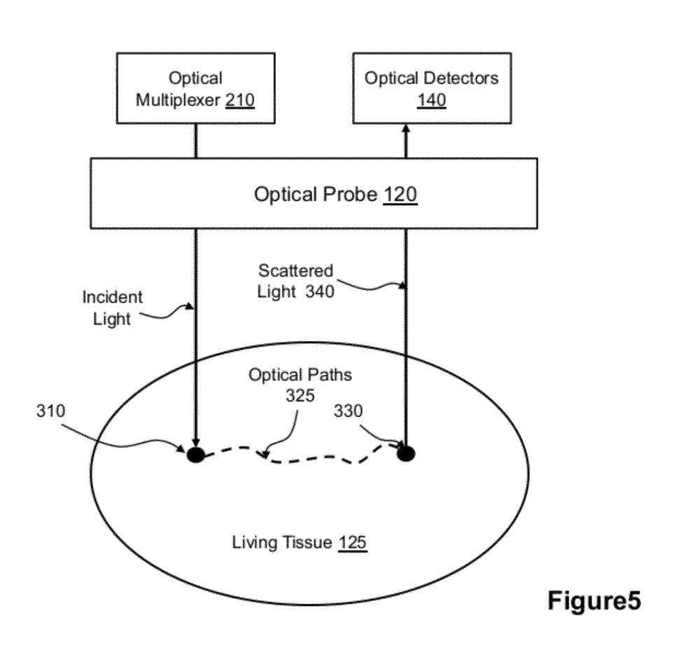

An important and advantageous feature of the present application is that self-calibration (step 412) is done based on a single optical path in the living tissue instead of multiple optical paths. The single optical-path calibration is achieved by emitting two or more NIR lights each modulated by a different frequency at a single light emitting point and collected at a single light collection point (e.g. between the light entry point 310 and the light exiting point 330 in FIG. 3A, or between the light emitting point 351 and the light collecting point 362, etc.). The single optical path 325 is also illustrated in FIG. 5.

In some embodiments, the optical probe in the presently disclosed tissue oximeter system includes a single light emitting point and a single light collecting point, which can introduce NIR lights into a living tissue and detect scattered NIR lights through a single optical path. The NIR lights, as described above, are modulated at different radio frequencies.

To help understand how self-calibration can be achieved in a single optical path, without limit to the specific mathematical expression, the detected light intensity can be approximately represented as

Ic(f)=Io*Sc*g(f,d,ua,us) Eqn. (1)

where Ic(f) is intensity of the detected light at detecting point C; Io is the amplitude of original source intensity modulated by a RF wave; f is the frequency of the RF wave; d is the distance between the light entry point and the light exit point; Sc represents effect of sensitivity, coupling efficiency, noise, etc., on the detected light intensity; ua and us are absorption and scattering coefficients, respectively, g is a complicated function of the those factors.

In the presently disclosed system, each light emitting point or light entry point of the living tissue is introduced with at least two NIR lights each modulated at a different frequency, for example, f1, f2. From, Eqn. (1), we have:

Ic(f1)/Ic(f2)=g(f1,d,ua,us)/g(f2,d,ua,us) Eqn. (2)

which is not dependent on Io and Sc. Thus, multiple RF modulation allows variations, and manufacturing errors to be performed with a single optical path. The effects of optical and mechanical errors, parameter deviation of different devices, and parameter changes over time can be self-calibrated. In addition, electronic noise including intrinsic electronic and optoelectronic noise can be reduced or eliminated because the NIR light modulated by different RF frequencies travel the same optical path and share substantially the same noise. As a result, the effects of manufacturing errors in source-detector separation and other above described variations are reduced to less than half.

Furthermore, a single optical path also allows smaller optical probe for tissue oximeters applicable to neonatal cerebral clinics, with more accuracy and precision in bio-information such as total hemoglobin, tissue oxygenation and glucose. The presently disclosed optical probe 120 (FIG. 1) can be made much smaller than those in conventional systems, which reduces the effects of inhomogeneities.

In general, the number of the optical detectors 140 (FIG. 1) can be different from the M number of NIR light sources (FIG. 1). In some embodiments, to simplify the self single-optical-path based calibrations as described above, the MRFM unit 110 (FIG. 2) can utilize the same numbers of light sources 1-M and the optical detectors 140 (FIG. 2).

The above described single-optical-path based calibrations solve two challenging problems that have been unsolved exist in the NIRS and tissue oximeters utilizing multiple optical paths: one problem relates to uncertainty of physical tolerance error in manufacturing the optical probes which dominate the accuracy of optical path length. The uncertainty of optical path lengths leads to incorrect data to calculate the optical properties and therefore incorrect bio-information about hemoglobin. The other problem is that the optical probes become too large when the optical probes need to accommodate multiple light emitting and light collecting points for multiple optical paths. The increased probe sizes increases the magnitudes of manufacturing errors, and is inconvenient for detecting small area of living tissue such as brain oxygenation for the newborns.

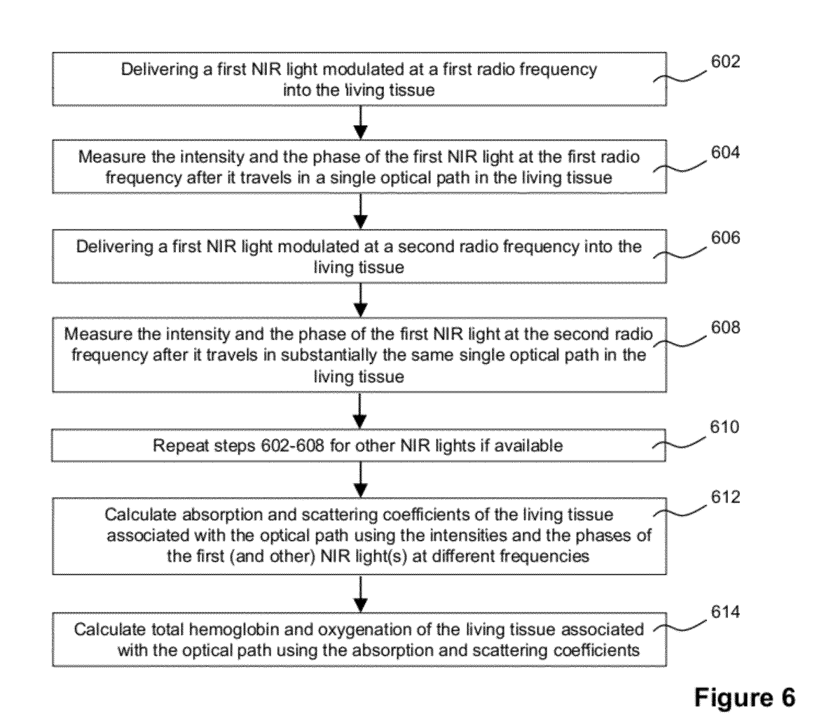

Referring to FIG. 6 and equations (1) and (2) above, the self-calibration of the tissue oximeter system can include the following steps in accordance with the present invention: a first NIR light modulated at a first radio frequency is delivered into a single optical path in the living tissue (step 602). The single optical path, as illustrated in FIG. 5, includes a single light entry point 310 and a single light exit point 330. The intensity and the phase of the first NIR light modulated are measured after it travels through a single optical path in the living tissue (step 604). The first NIR light modulated at a second radio frequency is delivered into the living tissue along the same single optical path (step 606). The light entry point and the light exit point can be kept the same when the NIR light is switched. The intensity and the phase of the first NIR light are measured after it travels through substantially the same single optical path in the living tissue (step 608). The steps 602-608 are repeated for a second NIR light (or the other NIR lights) if available (step 610). The absorption and scattering coefficients of the living tissue associated with the optical path are calculated using the intensities and the phases of the first and the other NIR lights at different frequencies (step 612). The calculations involve a ratio of the light intensities of the two NIR lights measured at the light collection point (light exit point), similar to what's shown in Equation (2). The calculations can also involve calculating a phase difference between the light intensities of the two NIR lights. As discussed above, the phase shifts of the scattered NIR lights reflect optical and thus biological properties of the living tissue along the optical path. The total hemoglobin and oxygenation of the living tissue associated with the optical path can be calculated using the absorption and scattering coefficients (step 614).

While this document contains many specifics, these should not be construed as limitations on the scope of an invention that is claimed or of what may be claimed, but rather as descriptions of features specific to particular embodiments. Certain features that are described in this document in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination.

For real time and noninvasive measurement, detection and monitor of absolute values of oxyhemoglobin and deoxyhemoglobin in blood volume and the living tissues in clinic and pathological practices and researches as well as healthcare. In the presently disclosed tissue oximeter system, the numbers of light source s and optical detectors can be minimized to reduce or eliminate the effects of tissue inhomogeneity in measuring the levels of oxyhemoglobin and deoxyhemoglobin.

Only a few examples and implementations are described. Other implementations, variations, modifications and enhancements to the described examples and implementations may be made without deviating from the spirit of the present invention. For example, the values for the numbers of light sources, optical detectors, and RF wave sources can be different from the examples described above. There can be the same number or different numbers of the light sources and the optical detectors.

1. A tissue oximeter system, comprising:
N number of radio frequency (RF) wave sources configured to produce RF waves at different RF frequencies, wherein N is an integer bigger than 1;
M number of near infrared (NIR) light sources each configured to emit NIR lights each modulated by one or more of the RF waves generated by the N number of RF wave sources, wherein M is an integer bigger than 1;
an optical probe configured to direct the NIR lights modulated at different RF frequencies to a living tissue, and wherein the optical probe comprises a plurality of light-emitting points each configured to couple one of the NIR lights into the living tissue;
one or more optical detectors each configured to receive scattered lights from the living tissue and to convert the scattered lights into electronic signals; and
a control and data acquisition unit configured to calculate absolute level of [HbO], [Hb], or [SO2] based on the electronic signals.

2. The tissue oximeter system of claim 1, further comprising:
an optical fiber bundle comprising a plurality of optical fibers each configured to deliver one of the NIR lights to the plurality of light-collecting points on the optical probe.

3. The tissue oximeter system of claim 1, further comprising:
an optical multiplexer configured to direct the NIR lights generated by the M number of NIR light sources to the optical probe; and
one or more optical fibers each configured to deliver one of the NIR lights from the optical multiplexer to the optical probe.

4. The tissue oximeter system of claim 3, wherein the optical multiplexer is configured to direct different ones of the NIR lights at different times through one of the one or more optical fibers.

5. The tissue oximeter system of claim 1, wherein the optical probe is configured to collect scattered lights from the living tissue at multiple locations on the living tissue, wherein the scattered lights have travelled through different optical paths in the living tissue.

6. The tissue oximeter system of claim 5, wherein the optical probe comprises a plurality of light-collecting points configured to collect the scattered lights at the multiple locations on the living tissue.

7. The tissue oximeter system of claim 1, wherein there are a different number of the optical detectors from the M number of NIR light sources.

8. The tissue oximeter system of claim 1, wherein there are M number of the optical detectors.

9. A tissue oximeter system, comprising:
a plurality of radio frequency (RF) wave sources configured to produce RF waves at different RF frequencies;
one or more near infrared (NIR) light sources each configured to emit NIR lights each modulated by one or more the RF waves at different RF frequencies;
an optical probe configured to direct the NIR lights modulated at different RF frequencies to a living tissue;
one or more optical detectors configured to receive scattered lights from the living tissue and to convert the scattered lights into electronic signals; and
a control and data acquisition unit configured to calculate absolute level of [HbO], [Hb], or [SO2] based on the electronic signals.

10. The tissue oximeter system of claim 9, wherein the scattered lights have travelled in different optical paths in the living tissue.

11. The tissue oximeter system of claim 10, wherein the optical probe comprises a plurality of light-emitting points each configured to couple one of the NIR lights into the living tissue.

12. The tissue oximeter system of claim 10, wherein the optical probe comprises a plurality of light-collecting points configured to collect the scattered lights at the multiple locations on the living tissue.

13. The tissue oximeter system of claim 9, wherein the scattered lights have travelled in single optical path in the living tissue.

14. The tissue oximeter system of claim 9, wherein the optical probe comprises a single light-emitting point configured to couple each of the NIR lights into the living tissue and a single light-collecting point configured to collect the scattered lights after the NIR lights travels through the single optical path.

15. A method for calibrating a tissue oximeter system, comprising:
modulating a first near infrared (NIR) light at a first radio frequency at a first light source;
delivering the first NIR light at the first radio frequency in a single optical path in a living tissue;
measuring intensity and phase of the first NIR light at the first radio frequency after the first NIR light travels through the single optical path in the living tissue;
modulating the first NIR light at a second radio frequency at the first light source;
delivering the first NIR light at the second radio frequency in the same single optical path in the living tissue;
measuring intensity and phase of the first NIR light at the second radio frequency after the first NIR light travels through the single optical path in the living tissue; and
calculating absorption coefficient and scattering coefficient of the living tissue using the intensities and the phases of the first NIR light at the first radio frequency and the first NIR light at the second radio frequency.

16. The method of claim 15, further comprising:
calculating total hemoglobin and oxygenation of the living using the absorption and scattering coefficients.

17. The method of claim 15, further comprising:
modulating a second NIR light at the first radio frequency at a second light source;
delivering the second NIR light at the first radio frequency in the single optical path in the living tissue;
measuring intensity and phase of the second NIR light at the first radio frequency after the second NIR light travels through the single optical path in the living tissue;
modulating the second NIR light at a second radio frequency at the second light source;
delivering the second NIR light at the second radio frequency in the same single optical path in the living tissue; and
measuring intensity and phase of the second NIR light at the second radio frequency after the second NIR light travels through the single optical path in the living tissue,
wherein the absorption coefficient and scattering coefficient of the living tissue are calculated in part using the intensities and the phases of the second NIR light at the first radio frequency and the second NIR light at the second radio frequency.

18. The method of claim 15, wherein the step of calculating comprises:
calculating a ratio of the light intensities of the first NIR light at the first radio frequency and the first NIR light at the second radio frequency measured after the first NIR light has travelled through the single optical path in the living tissue.

19. The method of claim 15, wherein the step of calculating comprises:
calculating a phase difference between the light intensities of the first NIR light at the first radio frequency and the first NIR light at the second radio frequency measured after the first NIR light has travelled through the single optical path in the living tissue.

20. The method of claim 15, wherein the single optical path is defined by a single light entry point and a single light exit point on the living tissue.
